(19) United States
(12) Patent Application Publication
(10) Pub. No.: US 2015/0094694 A1
(43) Pub. Date: Apr. 2, 2015

(54) Catheter Sterilization Cap

(71) Applicants: James W. Stone, Longmeadow, MA (US); Steven G. Richter, Southwick, MA (US)

(72) Inventors: James W. Stone, Longmeadow, MA (US); Steven G. Richter, Southwick, MA (US)

(21) Appl. No.: 14/043,466

(22) Filed: Oct. 1, 2013

(51) Int. Cl. A61M 39/16; A61M 39/10

(52) U.S. Cl. CPC A61M 39/162; A61M 39/1011; A61M 2039/1083; A61M 2039/1088
USPC 604/533

(57) ABSTRACT

A device for disinfecting and protection of a catheter thread end. The device consists of a main body with a inner sealing member and a outer cap that defines a cavity for the containment of a disinfecting agent. The disinfecting agent will be dispensed to cleanse the patient catheter threads when the catheter disinfecting cap is threaded to full engagement of the patient catheter.

### BACKGROUND OF THE INVENTION

This invention is for the administration of a disinfecting agent to sterilize the distal end catheter threads of a sterile cannula which may be surgically implanted in the body.

The catheter thread end, which is located outside the patient's body, is a convenient reusable sterile conduit for the daily exchange of intravenous feeding, transfusions, dialysis and medications. It is therefore essential that these connections be maintained in a sterile/aseptic condition. The present day practice of sterilizing the catheter threads is by swabbing them with an antiseptic coated gauze pad or swab which has the potential of allowing some of the antiseptic agent or contaminant to enter the conduit passageway and the patient. The Catheter Disinfecting Cap is designed to allow a controlled flow of the disinfecting agent to bathe the threads as the cap is threaded to full engagement while keeping the inner canal of the catheter protected from aseptic solution and or contamination.

### SUMMARY OF THE INVENTION

The device is to provide a method of supplying a quantity of disinfecting agent (Ethyl Alcohol & Saline Solution) to the distal end of the patient catheter threads and cleanse them from bacteria prior to attachment of any intravenous, dialysis, and or other types of medical implements.

The disinfecting Cap Body is attached to a patient catheter distal end threads by turning the Cap Body clockwise to full thread engagement. As the Cap Body engages the first thread of the patient catheter tube the Inner Sleeve will contact the catheter tube face and seal the catheter canal from exposure to any disinfecting agent during the cleansing process. A compression Spring will further keep the Inner Sleeve secure against the catheter face as the Cap Body continues engage more catheter threads. The internal grooves in the Cap Body will dispense the disinfecting agent, at a controlled flow rate, when the end of the grooves in the Cap Body is exposed to the cavity with the disinfecting agent and the Inner Sleeve.

The same device may remain attached to the patient catheter to prevent any microbial contamination from entering the distal end, of the catheter canal, when not in use.

### BRIEF DESCRIPTION OF THE DRAWINGS

FIG. 1 is a ‘3D’ model of the catheter sterilization cap, attached to a patient catheter with the Cap Body 10 in the full thread engagement and closed position.

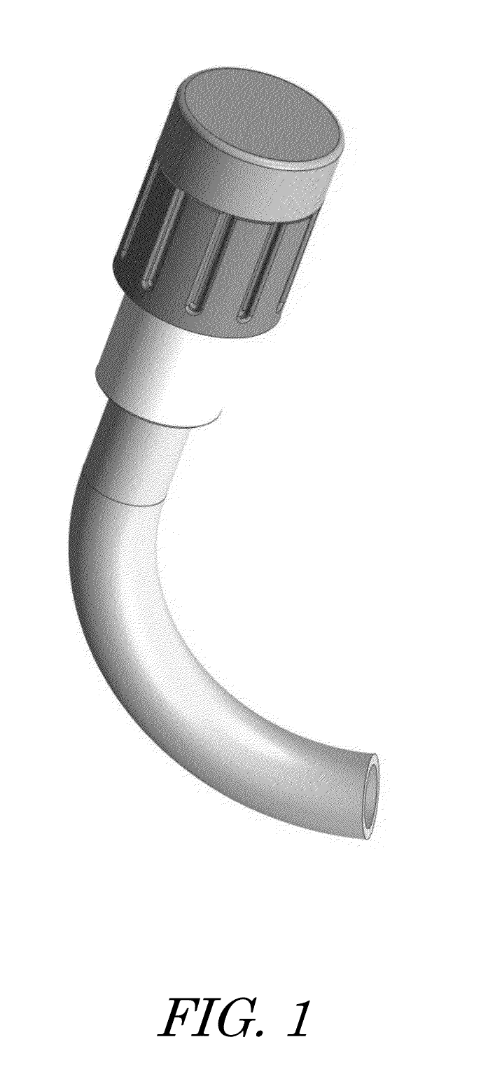

FIG. 2 is a cross section view of the catheter sterilization assembly with the Cap Body 10 engaging the patient catheter's first and second thread open position. Seal 50 between Cap Body 10 flange face and Inner Sleeve 20 flange face.

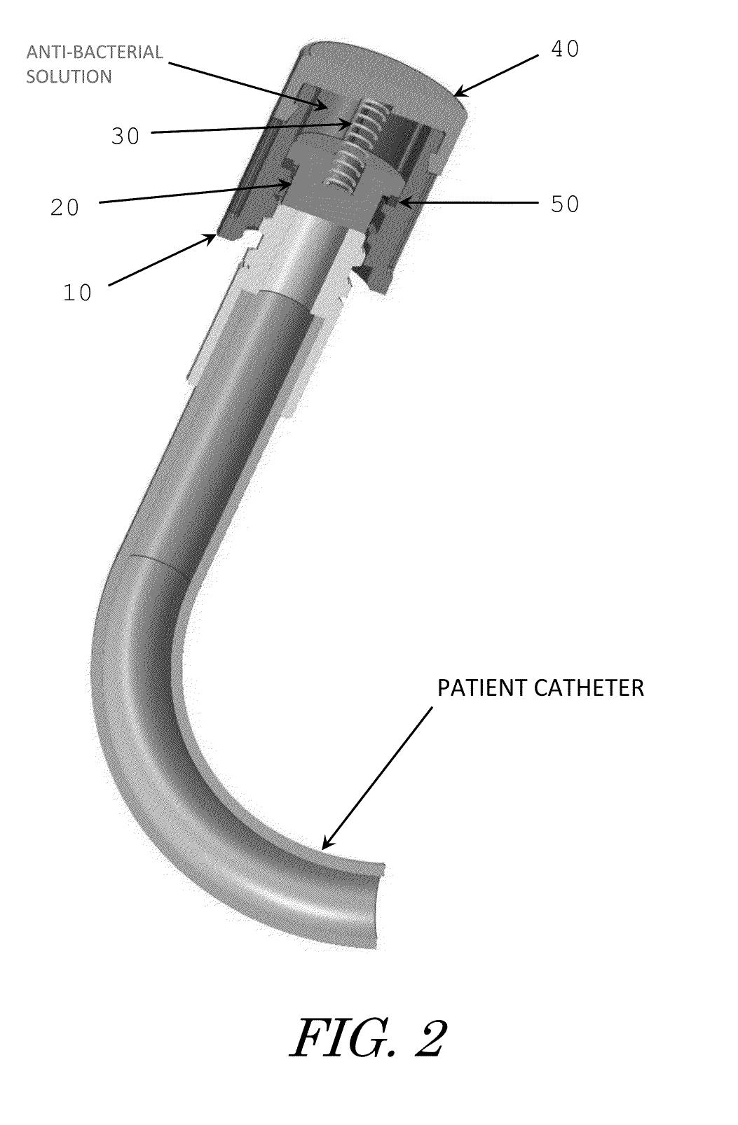

FIG. 3 is a cross section view of the catheter sterilization assembly with the Cap Body 10 in the full thread engagement (closed position).

FIG. 4 is an exploded view of all the parts 10, 20, 30, 40 & 50 of the catheter sterilization assembly.

FIG. 5 is an enlarged section view of the Cap Body 10 and the tapered drain slots that are designed to control the flow of the disinfecting agent to the catheter treads.

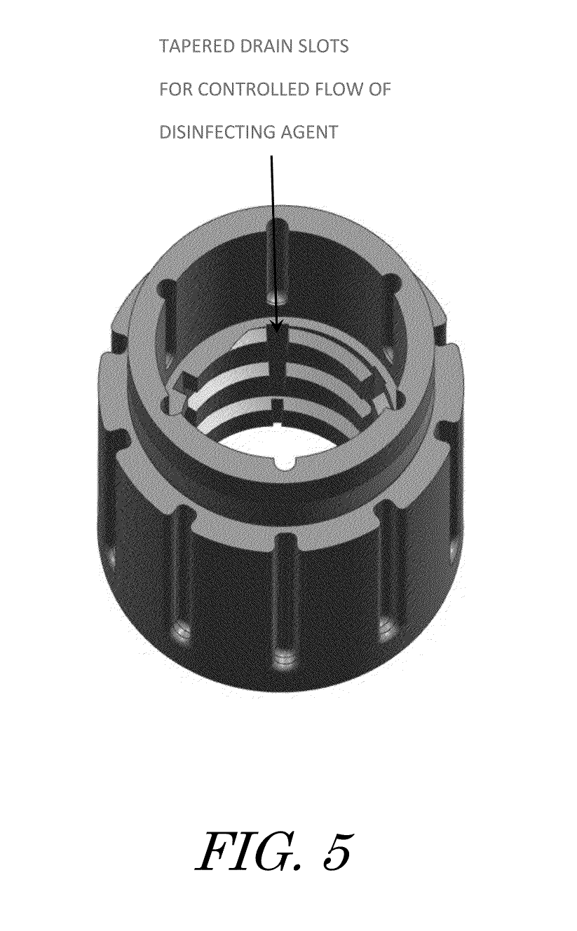

### DETAILED DESCRIPTION OF THE PREFERRED EMBODIMENT

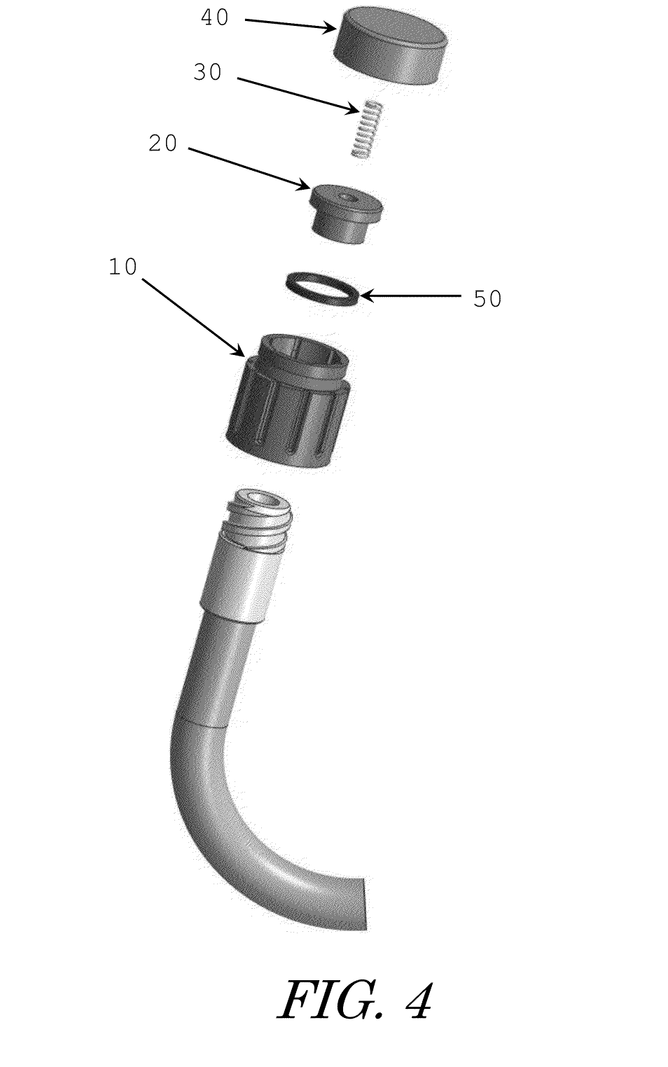

FIG. 4 illustrates the various components of the entire assembly with a designated number from 10 to 40.

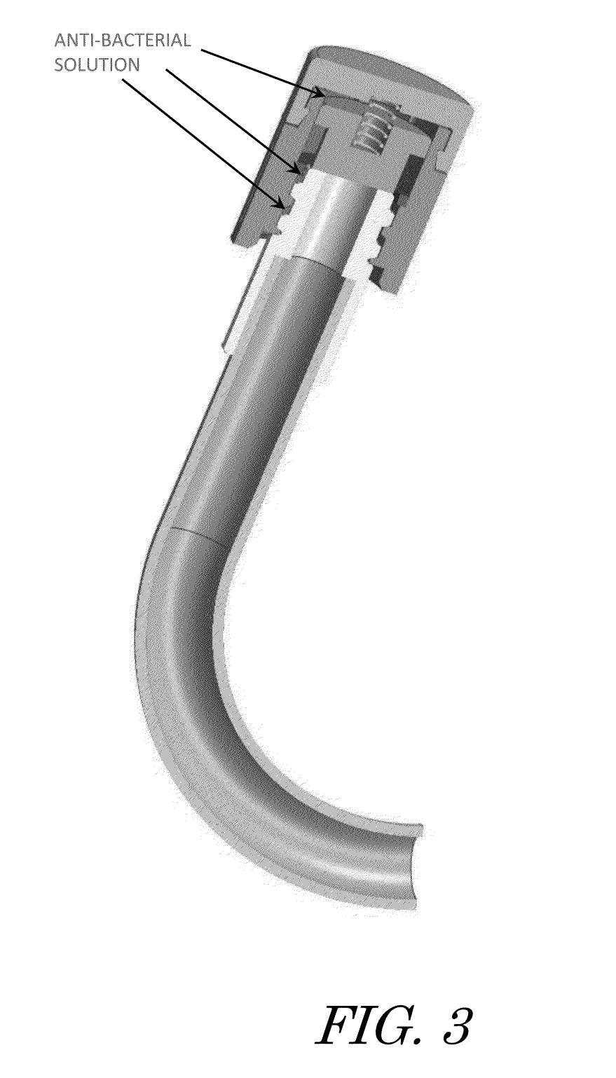

The main Cap Body 10 has female threads that are compatible with those of the patient catheter, male threads. It also houses the Inner Piston 20, Spring 30 and Seal 50 to contain the disinfecting agent from discharging until the main Body Cap 10 has been threaded to full closed position as shown in FIG. 3. The End Cap 40 is for ease of assembly, a liquid fill port and to provide reaction face for Spring 30 to maintain pressure against Inner Piston 20. The end face of the Inner Piston 20 prevents the disinfecting agent from entering the patient catheter canal.

What is claimed is:

1. A Catheter Disinfecting Cap comprising of a main body, inner sleeve, spring and end cap to contain the disinfecting agent in a cavity created by the inner sleeve in the bore of the main body and the end cap when attached to top of the main body until the main body is threaded onto a patient catheter at which time the disinfecting agent will be dispensed a at a controlled rate and cleanse the threads of the patient catheter.

2. The Catheter Disinfecting Cap according to claim 1, Wherein said main body of PVC material consists of a male Luers thread that is compatible with the female Luers thread of the patient catheter.

3. The Catheter Disinfecting Cap according to claim 2, Wherein said main body further consists of tapered drain slots for the controlled flow of the disinfecting agent when the main body is threaded on the patient catheter.

4. The Catheter Disinfecting Cap according to claim 2, Wherein said main body further consists of a barb type groove for locking and sealing of the end cap member.

5. The Catheter Disinfecting Cap according to claim 1, Wherein said second member inner sleeve of PVC material consists of a stepped diameter to retain and seal the disinfecting agent inside the main body cavity.

6. The Catheter Disinfecting Cap according to claim 1, Wherein said third member end cap of PVC material consists of a locking male barb that is captured in the barb groove of the main body when pressed on at assembly.

7. The Catheter Disinfecting Cap according to claim 1, wherein said fourth member spring made of stainless steel is the feature that maintains pressure on the inner sleeve by reacting of the end cap to seal both members and contain the disinfecting agent until the assembly is attached to the patient catheter.
